(19) United States
(12) Patent Application Publication
(10) Pub. No.: US 2023/0326561 A1
(43) Pub. Date: Oct. 12, 2023

(54) CLINICAL TRIAL SYSTEM, MOBILE TERMINAL, BLOOD TEST DEVICE, AND CLINICAL TRIAL SERVER

(71) Applicant: FUJIFILM CORPORATION, Tokyo (JP)

(72) Inventor: Keiji TSUBOTA, Tokyo (JP)

(21) Appl. No.: 18/175,553

(22) Filed: Feb. 28, 2023

(30) Foreign Application Priority Data
Mar. 25, 2022 (JP) 2022-049750

(51) Int. Cl. G16H 10/20; H04W 4/024

(52) U.S. Cl. CPC G16H 10/20; H04W 4/024

(57) ABSTRACT

A clinical trial system includes: a mobile terminal possessed by a trial subject; a blood test device installed in each of a plurality of testing facilities capable of conducting a blood test of the trial subject; and a clinical trial server that stores a test result of the trial subject acquired by the blood test device in association with trial subject information that specifies the trial subject. In the clinical trial system, route information to at least one testing facility capable of performing the blood test of the trial subject, which is retrieved on the basis of a clinical trial plan of the trial subject and position information of the trial subject, is displayed on the mobile terminal.

### CROSS REFERENCE TO RELATED APPLICATIONS

The present application claims priority from Japanese Patent Application No. 2022-049750, filed on Mar. 25, 2022, the entire disclosure of which is incorporated herein by reference.

### BACKGROUND

### Technical Field

The present disclosure relates to a clinical trial system, a mobile terminal, a blood test device, and a clinical trial server.

### Related Art

In order to obtain approval under the Pharmaceuticals and Medical Devices Act for the manufacture and sale of pharmaceuticals or medical devices, human clinical trials, that is, clinical trials, are being performed. In the clinical trial, a trial subject needs to perform blood collection at a predetermined time after taking medicine to be a clinical trial target. Therefore, various methods for reducing a burden on the trial subject who performs blood collection have been proposed. For example, WO2018/188909A proposes a method of providing a trial subject with a blood test kit, of registering an identification number assigned to the blood test kit in association with a value of the trial subject in a server, and of providing the trial subject with a recommended date and time for performing blood collection.

However, in the method disclosed in WO2018/188909A, it is necessary to send the collected blood to a testing institution. In the clinical trial, it is often necessary to perform blood collection a plurality of times during the day, and it is a heavy burden on the trial subject to perform a procedure of sending blood each time. For this reason, it is conceivable to hospitalize the trial subject for the test for the clinical trial, but it is also a heavy burden on the trial subject to keep the trial subject in the testing institution all day long. Further, the test date and time may extend over a plurality of days, and in such a case, the burden on the trial subject is further increased.

### SUMMARY OF THE INVENTION

The present invention has been made in view of the above circumstances, and an object of the present invention is to reduce a burden on a trial subject who has a blood test for a clinical trial.

According to the present disclosure, there is provided a clinical trial system comprising:
a mobile terminal possessed by a trial subject;
a blood test device installed in each of a plurality of testing facilities capable of conducting a blood test of the trial subject; and
a clinical trial server that stores a test result of the trial subject acquired by the blood test device in association with trial subject information that specifies the trial subject,
in which route information to at least one testing facility capable of performing the blood test of the trial subject is displayed on the mobile terminal, the testing facility being retrieved on the basis of a clinical trial plan of the trial subject and position information of the trial subject.

In the clinical trial system according to the present disclosure, the clinical trial server may retrieve the at least one testing facility from the plurality of testing facilities on the basis of the position information of the trial subject, and transmit a retrieval result of the testing facility to the mobile terminal, and the mobile terminal may display the route information to the retrieved testing facility.

In addition, in the clinical trial system according to the present disclosure, the mobile terminal may retrieve the at least one testing facility from the plurality of testing facilities on the basis of the position information of the trial subject, and display the route information to the retrieved testing facility.

Further, in the clinical trial system according to the present disclosure, the position information of the trial subject may indicate a current position of the trial subject.

Further, in the clinical trial system according to the present disclosure, the retrieved testing facility may be a testing facility located within a predetermined range from a position indicated by the position information of the trial subject.

Further, in the clinical trial system according to the present disclosure, in a case where the testing facility is not retrieved within the predetermined range from the position indicated by the position information of the trial subject, a new clinical trial plan for the trial subject may be set in the clinical trial server, and the testing facility may be retrieved on the basis of the new clinical trial plan and the position information of the trial subject.

Alternatively, in the clinical trial system according to the present disclosure, in a case where the testing facility is not retrieved within the predetermined range from the position indicated by the position information of the trial subject, a person involved in a clinical trial may be notified to that effect.

The “person involved in a clinical trial” includes a clinical trial coordinator who makes a clinical trial plan or medical personnel. The medical personnel include a doctor, a nurse, and a medical worker related to the clinical trial.

Further, in the clinical trial system according to the present disclosure, the trial subject may be provided with information regarding handling of the blood test device installed in the retrieved testing facility, through the mobile terminal.

Further, in the clinical trial system according to the present disclosure, the test result of the trial subject and the trial subject information may be transmitted from the mobile terminal or from the blood test device to the clinical trial server,
a new testing facility may be retrieved in a case where there is an abnormality in the test result, and
the route information to the new testing facility may be displayed on the mobile terminal.

Further, in the clinical trial system according to the present disclosure, the test result of the trial subject and the trial subject information may be transmitted from the mobile terminal or from the blood test device to the clinical trial server, and the mobile terminal or the clinical trial server may settle a test cost on the basis of the trial subject information after the transmission of the test result.

Alternatively, in the clinical trial system according to the present disclosure, the test result of the trial subject and the trial subject information may be transmitted from the mobile terminal or from the blood test device to the clinical trial server, and the clinical trial server may provide the mobile terminal with settlement information for settling a test cost, after the transmission of the test result.

Further, in the clinical trial system according to the present disclosure, the trial subject may be notified of guidelines for a test, through the mobile terminal.

According to the present disclosure, there is provided a mobile terminal used in the clinical trial system according to the present disclosure.

According to the present disclosure, there is provided a blood test device used in the clinical trial system according to the present disclosure.

According to the present disclosure, there is provided a clinical trial server used in the clinical trial system according to the present disclosure.

According to the present disclosure, it is possible to reduce the burden on the trial subject who has the blood test for the clinical trial.

### BRIEF DESCRIPTION OF THE DRAWINGS

FIG. 1 is a diagram showing a schematic configuration of a clinical trial system according to an embodiment of the present disclosure.

FIG. 2 is a diagram showing a hardware configuration of a mobile terminal according to the present embodiment.

FIG. 3 is a diagram showing a hardware configuration of a blood test device according to the present embodiment.

FIG. 4 is a diagram showing a hardware configuration of a clinical trial server according to the present embodiment.

FIG. 5 is a flowchart showing processing performed in the present embodiment.

FIG. 6 is a diagram showing a display screen of a list of retrieval results.

FIG. 7 is a diagram showing a display screen of route information.

FIG. 8 is a diagram showing a display screen of guidelines.

### DETAILED DESCRIPTION

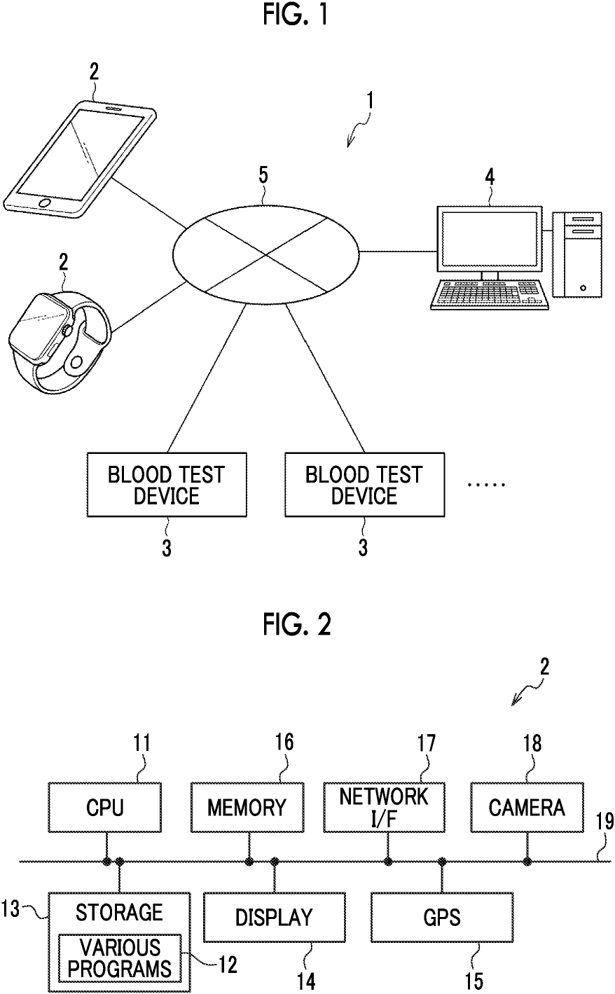

Hereinafter, an embodiment of the present disclosure will be described with reference to the drawings. FIG. 1 is a diagram showing a schematic configuration of a clinical trial system according to the present embodiment. As shown in FIG. 1, a clinical trial system 1 according to the present embodiment is a system for performing a blood test of a trial subject to be a clinical trial target, and has a mobile terminal 2 of the trial subject, blood test devices 3 installed in a plurality of testing facilities, and a clinical trial server 4 that are communicably connected to each other via a network 5. The clinical trial in the present embodiment includes not only a clinical trial for obtaining approval under the Pharmaceuticals and Medical Devices Act but also a clinical trial for obtaining efficacy and safety test data which is required for notification of foods for specific health use and products with function claims.

The mobile terminal 2 is a smartphone, a wearable terminal (a smartwatch), or the like possessed by the trial subject to be the clinical trial target. FIG. 2 is a diagram showing a hardware configuration of the mobile terminal. The mobile terminal 2 is a mobile computer, and comprises a central processing unit (CPU) 11, a non-volatile storage 13, and a memory 16 serving as a temporary storage area, as shown in FIG. 2. In addition, the mobile terminal 2 comprises a touch panel type display 14, a Global Positioning System (GPS) 15, and a network I/F 17 that is wirelessly connected to the network 5. In addition, the mobile terminal 2 comprises a camera 18. The CPU 11, the storage 13, the display 14, the GPS 15, the memory 16, the network I/F 17, and the camera 18 are connected to a bus 19.

The storage 13 is realized by a solid state drive (SSD), a flash memory, or the like. Various programs 12 installed on the mobile terminal 2 are stored in the storage 13 serving as a storage medium. The CPU 11 reads out the necessary program 12 from the storage 13 and then develops the read-out program 12 into the memory 16, and executes the developed program 12. Examples of the program 12 include programs, such as a map application, a clinical trial application, and a settlement application. Processing performed by the program 12 will be described below. The CPU 11 is an example of a processor that executes the program 12.

The various programs 12 are stored in a server (not shown) in a state of being accessible from an outside, and are downloaded and installed on the mobile terminal 2 in response to a request.

The display 14 includes a liquid crystal display, an organic EL, or the like, and performs various types of display related to processing performed by the mobile terminal 2. Further, the display 14 also has a function as an input device for performing various types of input to the mobile terminal 2.

The GPS 15 measures a current position of the mobile terminal 2 to derive position information. The derived position information is transmitted to the clinical trial server 4.

The camera 18 images a test result displayed or printed out by the blood test device 3, which will be described below, in response to an instruction from the trial subject. The test image acquired in this manner is stored in the storage 13 and is transmitted to the clinical trial server 4 in response to an instruction from the trial subject or without waiting for the instruction.

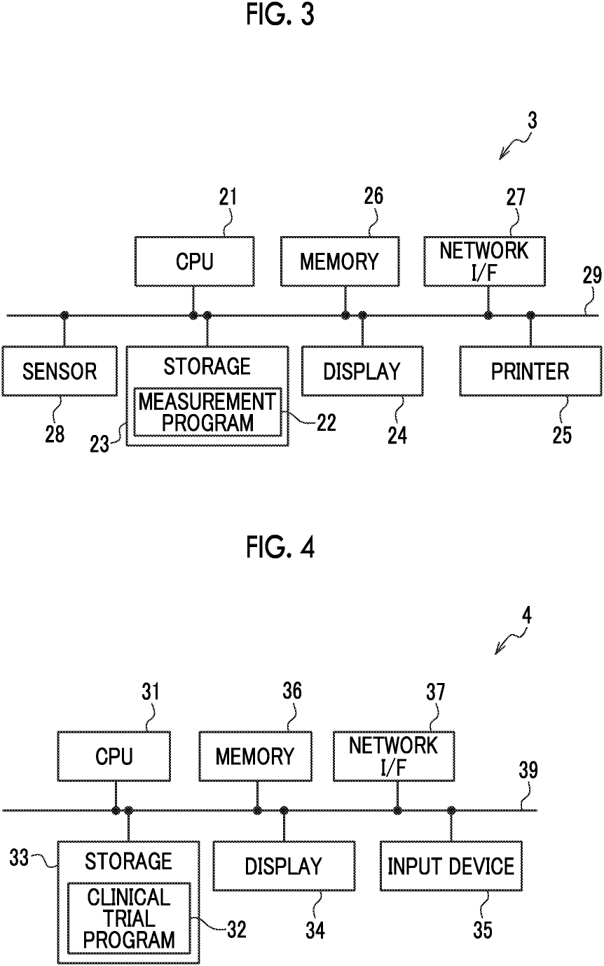

Next, the blood test device 3 will be described. The blood test device 3 is installed in the testing facility that performs a test for the clinical trial, such as a pharmacy or a hospital. FIG. 3 illustrates a hardware configuration of the blood test device 3. As shown in FIG. 3, the blood test device 3 comprises a CPU 21, a non-volatile storage 23, and a memory 26 serving as a temporary storage area. In addition, the blood test device 3 comprises a touch panel type display 24, a printer 25, a network I/F 27 that is connected to the network 5, and a sensor 28. The CPU 21, the storage 23, the display 24, the printer 25, the memory 26, the network I/F 27, and the sensor 28 are connected to a bus 29.

The storage 23 is realized by an SSD, a flash memory, or the like. The storage 23 serving as a storage medium stores a measurement program 22 for measuring biological information on blood, which is installed on the blood test device 3. The CPU 21 reads out the measurement program 22 from the storage 23 and then develops the read-out measurement program 22 into the memory 26, and executes the developed measurement program 22. The CPU 21 is an example of a processor that executes the measurement program 22.

The display 24 includes a liquid crystal display, an organic EL, or the like, and performs various types of display related to the processing performed by the blood test device 3 and display of the test result. In addition, the display 24 is a touch panel type display, and also has a function as an input device for performing various types of input to the blood test device 3.

The printer 25 prints out the test result on a recording medium, such as paper.

The sensor 28 measures biological information about the collected blood. For example, in a case where the biological information is a blood glucose level, the sensor 28 measures the blood glucose level. The blood collection may be performed by the trial subject through self-collection of blood using a blood collection kit, or may be performed by a nurse in a case where a nurse is present at the testing facility. The self-collection of blood is blood collection performed by the trial subject through a prick of a finger or the like by himself/herself.

The measurement program 22 is stored in a server (not shown) in a state of being accessible from the outside, and is downloaded and installed on the blood test device 3 in response to a request.

Next, the clinical trial server 4 will be described. The clinical trial server 4 is a server computer, and a clinical trial program for performing the clinical trial is installed on the clinical trial server 4. The clinical trial program is stored in a storage device of a server computer connected to the network or in a network storage in a state of being accessible from the outside, and is downloaded and installed on the clinical trial server 4 in response to a request. Alternatively, the clinical trial program is distributed by being recorded on a storage medium, such as a digital versatile disc (DVD) or a compact disc read only memory (CD-ROM), and is installed onto the clinical trial server 4 from the storage medium.

FIG. 4 is a diagram showing a hardware configuration of the clinical trial server. As shown in FIG. 4, the clinical trial server 4 includes a CPU 31, a non-volatile storage 33, and a memory 36 serving as a temporary storage area. In addition, the clinical trial server 4 includes a display 34, such as a liquid crystal display, an input device 35, such as a keyboard and a mouse, and a network I/F 37 that is connected to the network 5. The CPU 31, the storage 33, the display 34, the input device 35, the memory 36, and the network I/F 37 are connected to a bus 39.

The storage 33 is realized by an HDD, an SSD, a flash memory, or the like. A clinical trial program 32 is stored in the storage 33 serving as a storage medium. The CPU 31 reads out the clinical trial program 32 from the storage 33 and then develops the read-out clinical trial program 32 into the memory 36, and executes the developed clinical trial program 32. The CPU 31 is an example of a processor that executes the clinical trial program 32.

In addition, the storage 33 stores, as a database, information necessary for the clinical trial, such as information on the plurality of testing facilities participating in the clinical trial, information on types of tests that can be performed at each of the testing facilities (for example, a blood glucose level test, a lipid test, and a uric acid level test), handling information of the blood test device installed in each of the testing facilities, trial subject information for specifying a trial subject participating in the clinical trial (name, age, sex, pre-existing disease, and the like), and a clinical trial plan that determines procedures for the clinical trial. In addition, the database may include information on the accuracy of the blood test device 3 installed in the testing facility, information on whether or not the blood test device 3 can communicate with the clinical trial server 4, and the like.

In the clinical trial, the clinical trial coordinator makes the clinical trial plan in advance. The clinical trial plan includes a time point of blood collection during the day. The clinical trial plan includes the time point of blood collection, for example, 9 o′clock, 12 o′clock, 15 o′clock, and 18 o′clock. In addition, the clinical trial plan also includes a type of blood test performed on the trial subject. Examples of the type of blood test include types of tests, such as measurement for a blood glucose level, measurement for a lipid, and measurement for a uric acid level. The clinical trial plan is stored in the storage 33 in association with the trial subject.

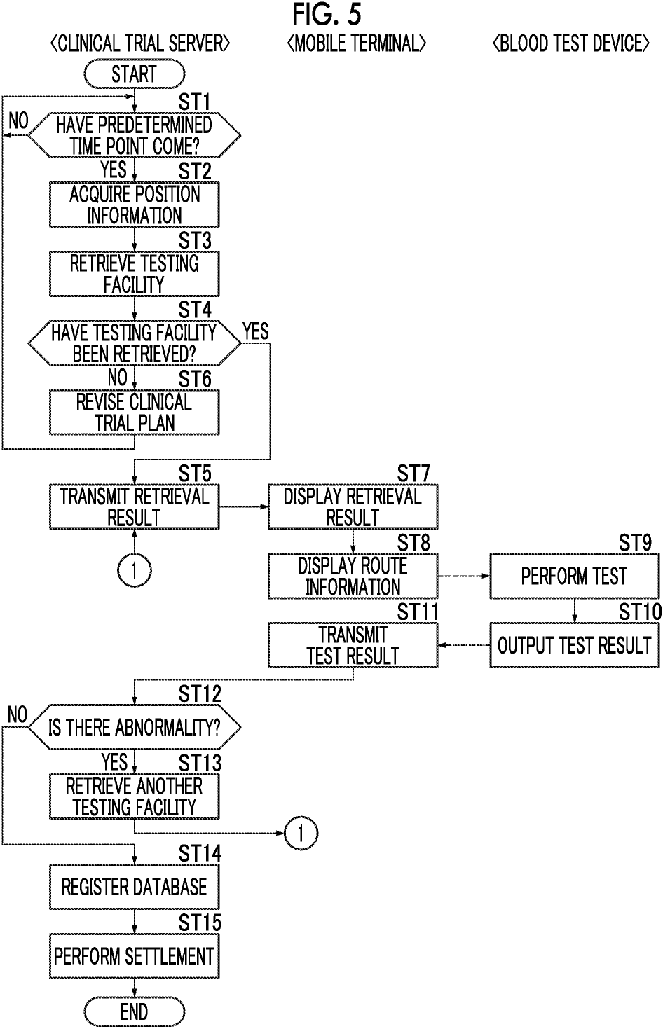

Next, processing performed in the clinical trial system 1 according to the present embodiment will be described. FIG. 5 is a flowchart showing the processing performed in the clinical trial system 1 according to the present embodiment. The flowchart shown in FIG. 5 shows the processing of the clinical trial for a certain trial subject. First, the clinical trial server 4 starts monitoring whether or not a predetermined time point before the time point of blood collection determined on the basis of the clinical trial plan of the trial subject has come (step ST1). The predetermined time point need only be set on the clinical trial server 4 by presenting candidates, such as 60 minutes, 30 minutes, 15 minutes, or the like before the time point of blood collection, to the trial subject in advance. Alternatively, it is also possible to set any time point desired by the trial subject.

In a case where affirmative determination is made in step ST1, the clinical trial server 4 acquires the position information indicating the current position of the trial subject from the mobile terminal 2 of the trial subject (step ST2). The clinical trial server 4 retrieves a testing facility that can perform the blood test of the trial subject and that is located within a predetermined range with the current position of the trial subject as a reference, on the basis of the acquired position information and the clinical trial plan of the trial subject (step ST3). The predetermined range need only be a range of distance in which the trial subject can arrive at the testing facility from the current position, from the current time point to the time point of blood collection. At this time, information on the means of transportation (walking, bicycle, automobile, or the like) of the trial subject may be transmitted from the mobile terminal 2 to the clinical trial server 4, and the testing facility may be retrieved in consideration of the information on the means of transportation. Further, the retrieval for the testing facility is performed so as to include a testing facility capable of conducting all blood tests performed on the trial subject.

The clinical trial server 4 determines whether or not the testing facility capable of performing the blood test of the trial subject has been retrieved (step ST4), and in a case where affirmative determination is made in step ST4, the clinical trial server 4 transmits the retrieval result of the testing facility to the mobile terminal 2 (step ST5).

In a case where negative determination is made in step ST4, the blood test cannot be performed in accordance with the clinical trial plan. Therefore, in a case where negative determination is made in step ST4, the clinical trial server 4 revises the clinical trial plan, sets the test time point and the like again (step ST6), and returns to the processing of step ST1. The clinical trial plan may be revised by the clinical trial program installed on the clinical trial server 4, or the clinical trial coordinator in charge of the relevant trial subject may revise the clinical trial plan and set the clinical trial plan in the clinical trial server 4. At this time, an administrator of the clinical trial server 4 need only retrieve the clinical trial coordinator in charge of the relevant trial subject and contact the retrieved clinical trial coordinator. The administrator of the clinical trial server 4 transmits the information on the trial subject, the current position information of the trial subject, the clinical trial plan, and the like to the clinical trial coordinator. In a case where negative determination is made in step ST4, the clinical trial server 4 may notify the clinical trial coordinator through e-mail or the like that the blood test cannot be performed in accordance with the clinical trial plan. The clinical trial coordinator revises the clinical trial plan on the basis of this notification and transmits the revised clinical trial plan to the clinical trial server 4. In a case where negative determination is made in step ST4, a doctor, a nurse, or another person involved in the clinical trial, who is a medical worker related to the clinical trial, may be notified instead of the clinical trial coordinator.

In a case where the clinical trial server 4 has revised the clinical trial plan, it is preferable that the clinical trial server 4 transmits information to that effect to the mobile terminal 2 to notify the trial subject. Alternatively, in a case where the clinical trial coordinator has revised the clinical trial plan, it is preferable that the clinical trial coordinator contacts the trial subject and notifies the trial subject that the clinical trial plan has been changed.

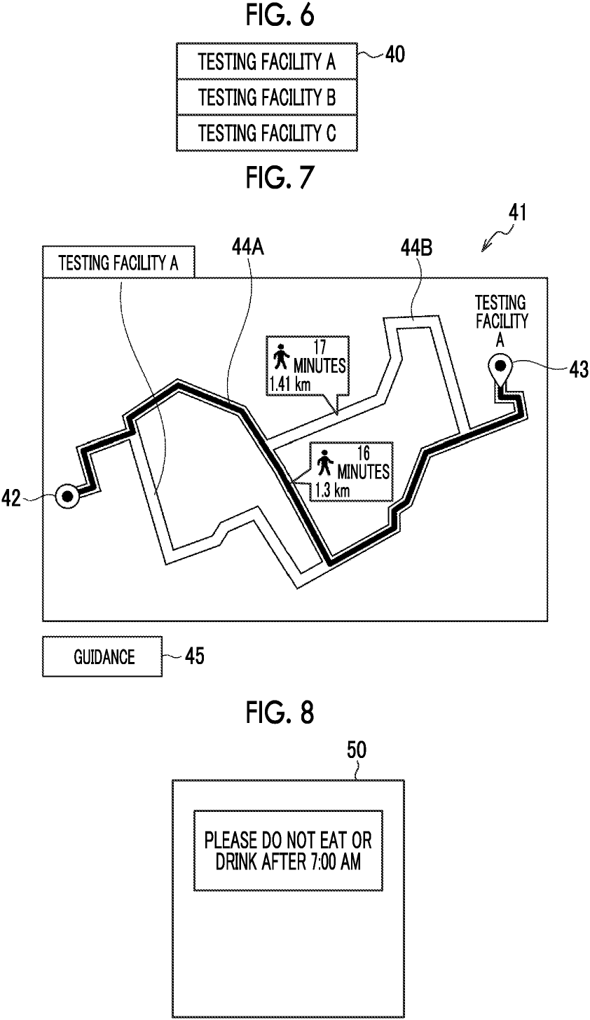

The mobile terminal 2 displays the retrieval result of the testing facility on the display 14 (step ST7). FIG. 6 is a diagram showing the retrieval result of the testing facility. As shown in FIG. 6, in a retrieval result 40, three testing facilities A to C are displayed as an example. In a case where the trial subject selects a desired testing facility in the retrieval result displayed on the mobile terminal 2, the mobile terminal 2 displays route information from the current position of the trial subject to the selected testing facility on the display 14 (step ST8).

FIG. 7 is a diagram showing the route information to the selected testing facility. As shown in FIG. 7, in route information 41, a mark 42 indicating the current position of the trial subject, a mark 43 indicating the selected testing facility, and two routes 44A and 44B from the current position of the trial subject to the selected testing facility are displayed. It is possible to display a route to another testing facility by reselecting the testing facility. The trial subject can move to the selected facility in accordance with a desired route among the displayed routes 44A and 44B.

In addition, a guidance button 45 is displayed on the display 14 of the mobile terminal 2 together with the route information 41. The trial subject selects the guidance button 45 so that information regarding the handling of the blood test device 3 installed in the selected testing facility is provided to the trial subject by being displayed on the display 14. The information regarding the handling includes a test method in the blood test device 3 and guidelines when the test is performed. The display may be a text for sequentially describing the test method, or still images sequentially showing the test method or a motion picture including an animation. Instead of or in addition to the display, the information regarding the handling of the blood test device 3 may be provided to the trial subject by voice.

In addition, the trial subject may not understand a content of guidance. Therefore, a button for contacting the clinical trial coordinator may be displayed on a display screen that displays the guidance. In this case, the trial subject can contact the clinical trial coordinator from the mobile terminal 2 through telephone, e-mail, or the like by operating the button, and the clinical trial coordinator can directly give a lecture on the guidance of the blood test device 3. Instead of the guidance given by the clinical trial coordinator, the guidance may be given by a machine voice.

The clinical trial program may be installed on the mobile terminal 2, and the processing of steps ST1 to ST6 may be performed in the mobile terminal 2. In this case, in a case where negative determination is made in step ST4, information indicating that the testing facility could not be retrieved is displayed on the display 14 instead of the list of retrieval results. Further, the trial subject notifies the clinical trial server 4 to that effect, through the mobile terminal 2. The clinical trial server 4 revises the clinical trial plan, sets the test time point and the like again, and transmits the set test time point to the mobile terminal 2. The mobile terminal 2 need only retrieve the testing facility again in accordance with the set test time point. This processing need only be performed until the testing facility is retrieved.

In addition, a contact button for contacting the clinical trial coordinator may be displayed on the mobile terminal 2, in addition to the display of the information indicating that the testing facility could not be retrieved. In this case, the trial subject need only have the clinical trial plan revised by selecting the contact button to call the clinical trial coordinator.

The trial subject who has moved to the testing facility performs self-collection of blood by using a blood collection kit in the testing facility, or asks a doctor or a nurse in the testing facility to perform blood collection. Then, the collected blood is set in the blood test device 3, and an instruction to start the test is given to the blood test device 3. With this, the blood test device 3 performs the test (step ST9) and outputs the test result (step ST10). The test result may be output by being displayed on the display 24 or by being printed out from the printer 25.

The trial subject images the output test result with the camera 18 of the mobile terminal 2, and transmits the captured image together with the trial subject information specifying the trial subject to the clinical trial server 4 (test result transmission: step ST11). Communication may be performed between the mobile terminal 2 and the blood test device 3 such that the mobile terminal 2 can receive the test result from the blood test device 3. In this case, the mobile terminal 2 transmits the test result received from the blood test device 3 together with the trial subject information to the clinical trial server 4. Alternatively, the blood test device 3 performs communication with the clinical trial server 4, whereby the blood test device 3 may transmit the test result to the clinical trial server 4. At this time, the blood test device 3 transmits, to the clinical trial server 4, the test result and the trial subject information on the trial subject who had the test.

Upon receiving the test result, the clinical trial server 4 determines whether or not there is an abnormality in the test result (step ST12). The term “abnormality” as used herein does not mean that the test result deviates from a reference value, and means that a preposterous test result is obtained because of a failure of the blood test device 3, a measurement error, or the like. A result of the past blood test of the trial subject may be stored in the clinical trial server 4, and whether or not there is an abnormality may be determined by referring to the result. In addition, the abnormality also includes a case where the test result is not acquired. Instead of determining whether or not there is an abnormality in the test result, for example, the blood test device 3 may transmit information indicating an abnormality, such as an error code, to the clinical trial server 4 in a case where the blood test device 3 detects the abnormality, and the clinical trial server 4 may determine that there is an abnormality in the test result on the basis of the information received from the blood test device 3.

In a case where affirmative determination is made in step ST12, the clinical trial server 4 retrieves another testing facility (step ST13) and returns to the processing of step ST5. With this, the retrieval result of another testing facility is transmitted to the mobile terminal, the other testing facility is displayed on the mobile terminal 2, and as a result, the trial subject is urged to have a retest. At that time, it is preferable to also present to the mobile terminal 2 that there is an abnormality in the test result. The trial subject can have the test by moving to the other testing facility. In a case where a plurality of test devices are provided in the same testing facility, another test device in the same testing facility may be retrieved. In that case, the trial subject need only have the test by using the other test device in the same testing facility without moving.

In a case where negative determination is made in step ST12, the clinical trial server 4 registers the test result in association with the trial subject information in the database (step ST14).

Here, a test cost is incurred for the test performed at the testing facility. The test cost is borne by a testing institution that performs the clinical trial and does not need to be paid by the trial subject. In this case, the trial subject can also pay the test cost at the testing facility on behalf of the testing institution. Although it is temporary, the trial subject bears a financial burden, and a burden on the trial subject is heavy because it is necessary to perform processing, such as charging the cost later.

Therefore, the clinical trial server 4 settles the test cost on the basis of the trial subject information (step ST15), and ends the processing. The mobile terminal 2 and the facility in which the blood test device 3 is installed may be notified that the settlement has been completed and the test has ended. Instead of the clinical trial server 4, the clinical trial application installed on the mobile terminal 2 may perform the settlement on the mobile terminal 2.

Instead of settling the test cost on the clinical trial server 4, settlement information for the settlement may be provided to the mobile terminal 2 after step ST14. As the settlement information, a two-dimensional bar code, such as a QR code (registered trademark), can be used. In this case, the trial subject displays the two-dimensional bar code on the mobile terminal 2 and presents the two-dimensional bar code at the cash register of the testing facility that has performed the test to settle the test cost. Alternatively, the settlement information may be information for notifying Near Field Communication (NFC) of the mobile terminal 2 of payment permission. In this case, the trial subject performs the settlement at the cash register of the testing facility by using the NFC of the mobile terminal 2.

As described above, in the present embodiment, the route information to at least one testing facility capable of performing the blood test of the trial subject, which is retrieved on the basis of the clinical trial plan of the trial subject and the position information of the trial subject, is displayed on the mobile terminal 2. Therefore, the trial subject can have the blood test for the clinical trial by moving to the testing facility in accordance with the route information.

As a result, the trial subject can have the blood test without sending the collected blood to the testing institution or without being hospitalized for the test and being kept in the testing institution all day long. Accordingly, it is possible to reduce the burden on the trial subject who has the test for the clinical trial. In addition, since it is easier for the testing institution to collect the trial subjects, population parameters of the clinical trial can be increased, and as a result, the reliability of the clinical trial can be improved. Further, since a transportation cost for the trial subject is not necessary even in a case where the testing institution is located in a remote place, the cost for the clinical trial can be reduced. Further, in a situation where infectious disease is widespread, waiting for a long time in a waiting room of the testing institution or staying at the testing institution all day long has a high risk of infection. However, according to the present embodiment, the risk of such infection can be reduced.

In the above-described embodiment, the testing facility is retrieved on the basis of the current position of the trial subject, but the present disclosure is not limited thereto. The trial subject may designate any place before the test day and retrieve the testing facility on the basis of the position information of the designated place, through the mobile terminal 2. For example, in a case where the trial subject is scheduled to go out on the test day, the trial subject may retrieve the testing facility on the basis of the position information of a destination on the day before the test day or while moving by train or the like on the day of the test. As a result, since the trial subject can know in advance at which testing facility the trial subject can have the test, it is easier to set an activity schedule for the test day.

In addition, in the above-described embodiment, the trial subject may be notified of the guidelines for the test, through the mobile terminal 2. For example, in a case where eating or drinking is prohibited before a predetermined time of the test time point, as shown in FIG. 8, guidelines 50 for the test, such as “Please do not eat or drink after 7:00 AM”, may be presented on the display 14 of the mobile terminal 2 before the predetermined time of the test time point. In this case, the presentation of the guidelines may be performed by the clinical trial program installed on the mobile terminal 2, or the clinical trial program 32 installed on the clinical trial server 4 may notify the mobile terminal 2 and the presentation of the guidelines may be performed by the mobile terminal 2 on the basis of the notification.

Further, in the above-described embodiment, for example, various processors shown below can be used as the hardware structure of a processing unit that executes various types of processing performed by the mobile terminal 2, the blood test device 3, and the clinical trial server 4. The above-described various processors include, for example, a programmable logic device (PLD) which is a processor having a changeable circuit configuration after manufacture, such as a field programmable gate array (FPGA), and a dedicated electrical circuit which is a processor having a dedicated circuit configuration designed to execute specific processing, such as an application specific integrated circuit (ASIC), in addition to the CPU which is a general-purpose processor that executes software (programs) to function as various processing units, as described above.

One processing unit may be composed of one of these various processors or a combination of two or more processors of the same type or different types (for example, a combination of a plurality of FPGAs or a combination of a CPU and an FPGA). Alternatively, a plurality of processing units may be composed of one processor.

A first example of the configuration in which the plurality of processing units are composed of one processor is an aspect in which one or more CPUs and software are combined to constitute one processor and the processor functions as a plurality of processing units, as represented by a computer, such as a client and a server. A second example of the configuration is an aspect in which a processor that realizes all the functions of a system including a plurality of processing units with one integrated circuit (IC) chip is used, as represented by a system on chip (SoC). As described above, various processing units are composed of one or more of the above-described various processors as the hardware structure.

Furthermore, as the hardware structure of the various processors, more specifically, an electrical circuit (circuitry) in which circuit elements, such as semiconductor elements, are combined can be used.

What is claimed is:

1. A clinical trial system comprising:
a mobile terminal possessed by a trial subject;
a blood test device installed in each of a plurality of testing facilities capable of conducting a blood test of the trial subject; and
a clinical trial server that stores a test result of the trial subject acquired by the blood test device in association with trial subject information that specifies the trial subject,
wherein route information to at least one testing facility capable of performing the blood test of the trial subject is displayed on the mobile terminal, the testing facility being retrieved on the basis of a clinical trial plan of the trial subject and position information of the trial subject.

2. The clinical trial system according to claim 1,
wherein the clinical trial server retrieves the at least one testing facility from the plurality of testing facilities on the basis of the position information of the trial subject, and transmits a retrieval result of the testing facility to the mobile terminal, and
the mobile terminal displays the route information to the retrieved testing facility.

3. The clinical trial system according to claim 1,
wherein the mobile terminal retrieves the at least one testing facility from the plurality of testing facilities on the basis of the position information of the trial subject, and displays the route information to the retrieved testing facility.

4. The clinical trial system according to claim 1,
wherein the position information of the trial subject indicates a current position of the trial subject.

5. The clinical trial system according to claim 1,
wherein the retrieved testing facility is a testing facility located within a predetermined range from a position indicated by the position information of the trial subject.

6. The clinical trial system according to claim 5,
wherein in a case where the testing facility is not retrieved within the predetermined range from the position indicated by the position information of the trial subject, a new clinical trial plan for the trial subject is set in the clinical trial server, and
the testing facility is retrieved on the basis of the new clinical trial plan and the position information of the trial subject.

7. The clinical trial system according to claim 5,
wherein in a case where the testing facility is not retrieved within the predetermined range from the position indicated by the position information of the trial subject, a person involved in a clinical trial is notified to that effect.

8. The clinical trial system according to claim 1,
wherein the trial subject is provided with information regarding handling of the blood test device installed in the retrieved testing facility, through the mobile terminal.

9. The clinical trial system according to claim 1,
wherein the test result of the trial subject and the trial subject information are transmitted from the mobile terminal or from the blood test device to the clinical trial server,
a new testing facility is retrieved in a case where there is an abnormality in the test result, and
the route information to the new testing facility is displayed on the mobile terminal.

10. The clinical trial system according to claim 1,
wherein the test result of the trial subject and the trial subject information are transmitted from the mobile terminal or from the blood test device to the clinical trial server, and
the mobile terminal or the clinical trial server settles a test cost on the basis of the trial subject information after the transmission of the test result.

11. The clinical trial system according to claim 1,
wherein the test result of the trial subject and the trial subject information are transmitted from the mobile terminal or from the blood test device to the clinical trial server, and
the clinical trial server provides the mobile terminal with settlement information for settling a test cost, after the transmission of the test result.

12. The clinical trial system according to claim 1,
wherein the trial subject is notified of guidelines for a test, through the mobile terminal.

13. A mobile terminal used in the clinical trial system according to claim 1.

14. A blood test device used in the clinical trial system according to claim 1.

15. A clinical trial server used in the clinical trial system according to claim 1.
